(19) United States
(12) Patent Application Publication
(10) Pub. No.: US 2026/0137541 A1
(43) Pub. Date: May 21, 2026

(54) MULTI-PURPOSE KNEE BRACE KIT AND ACCESSORIES THEREFOR

(71) Applicant: NUBRACE INNOVATIONS, INC., Brooklyn, NY (US)

(72) Inventor: Adoram LESHEM, Rishon Lezion (IL)

(21) Appl. No.: 19/450,450

(22) Filed: Jan. 15, 2026

Related U.S. Application Data
(63) Continuation-in-part of application No. PCT/IL2024/050697, filed on Jul. 16, 2024.

(30) Foreign Application Priority Data
Jul. 17, 2023 (IL) 304541

(51) Int. Cl. A61F 5/01; A63B 21/00; A63B 21/02; A63B 21/04; A63B 23/04

(52) U.S. Cl. CPC A61F 5/0123; A63B 21/026; A63B 21/0421; A63B 21/4011; A63B 23/0494; A61F 2005/0137; A61F 2005/0179

(57) ABSTRACT

Knee brace kits including a knee brace having a leg encircling arrangement for encircling the user's thigh and calf, a leg fastening arrangement for strapping the leg encircling arrangement on the user's thigh and calf, and a triple anchor arrangement pair mounted on the leg encircling arrangement wherein each triple anchor arrangement includes an opposite pair of a thigh anchor and a calf anchor, and a strut. Each strut includes a thigh strut member, a calf strut member, and a strut anchor therebetween for deployment at a knee's flexion/extension axis. Each strut anchor includes a bending resistance member holder. A bending resistance member is configured for longitudinal sliding insertion into an opposite pair of the thigh anchor and the calf anchor and mounting in the bending resistance member holder.

### CROSS-REFERENCE TO RELATED APPLICATIONS

This application is a Continuation-in-Part of International Application No PCT/IL 2024/050697, filed Jul. 16, 2024, which was published in the English language on Jan. 23, 2025, under International Publication No. WO 2025/017552A 1 , which claims priority under 35 U.S.C. § 119(b) to Israeli Application No. 304541, filed Jul. 17, 2023, the disclosures of each of which are incorporated herein by reference.

### FIELD OF THE INVENTION

The invention relates to knee braces.

### BACKGROUND OF THE INVENTION

Knee braces are disclosed in inter alia EP 0 941 722, EP 2 029 067, U.S. Pat. Nos. 8,343,083, 11,253,385, US 2009/0312683 and US 2020/0155405.

### SUMMARY OF THE INVENTION

The present invention is directed towards multi-purpose knee brace kits for assisting a user to straighten his leg and/or exercise his leg muscles. The knee brace kit includes a knee brace including a leg encircling arrangement for encircling a user's at least thigh and calf, and a leg fastening arrangement mounted on the leg encircling arrangement for strapping the leg encircling arrangement on a user's at least thigh and calf. The leg fastening arrangement is typically constituted by Velcro straps, tie laces, clips, and the like. The knee brace includes an outside leg triple anchor arrangement and/or an inside leg triple anchor arrangement for interchangeable mounting of one bending resistance member or a bending resistance member pair. Each triple anchor arrangement includes an opposite pair of a thigh anchor and a calf anchor, and a strut. A thigh anchor includes a thigh anchor blind bore facing a user's calf and a calf anchor includes a calf anchor blind bore facing a user's thigh. A strut includes a thigh strut member, a calf strut member, and a strut anchor between the thigh strut member and the calf strut member for deployment at a knee's flexion/extension axis. The strut anchor includes a bending resistance member holder. A bending resistance member is mounted in a triple anchor arrangement by longitudinal sliding insertion into an opposite pair of a thigh anchor and a calf anchor and insertion into a bending resistance member holder such that, in use on wearing a knee brace, a bending resistance member is lateral to a strut relative to a user's leg. A bending resistance member can be slid through a bending resistance member holder, snap fitted into a bending resistance member holder, and the like.

The triple anchor arrangements are designed to anchor a bending resistance member at its two opposite bending resistance member free ends and its center, thereby effectively ensuring a bending resistance member is maintained in a desired position relative to a user's leg during flexion/extension. Moreover, the triple anchor arrangements enable deployment of bending resistance members having a wide range of bending stiffnesses from 1 kg to 80 kg. The triple point anchor arrangement is particularly useful for bending resistance members of higher bending stiffnesses which would otherwise have a tendency to arch away from a user's leg, thereby detracting from their intended use.

Knee braces of the present invention can be designed to be optionally used with one stretch resistance member or a stretch resistance member pair instead of the one bending resistance member or the bending resistance member pair for enabling a user to perform leg flexion/extension exercises. The stretch resistance members are typically provided as stretch resistance bands, latex resistance tubes, and the like, and terminate at an opposite stretch resistance member free end pair constituted by a wide diameter stopper pair. The knee brace further includes a thigh fastening point proximal to a thigh anchor for removably fastening one stretch resistance member free end, and a calf fastening point distal to a calf anchor for removably fastening the other stretch resistance member free end whereby, on a user straightening his leg, a stretch resistance member is stretched mounted between the thigh fastening point and the calf fastening point. A thigh fastening point can be discrete from a thigh anchor or integrally formed therewith in the form of a thigh anchor transverse groove. Similarly, a calf fastening point can be discrete from a calf anchor or integrally formed therewith in the form of a calf anchor transverse groove.

Knee braces of the present invention can be constructed with different leg encircling arrangements as follows: A leg encircling arrangement can be constituted by a soft material wraparound intended to be wrapped around a user's leg before being strapped thereon, and reversibly removed from a user's leg. The soft material wraparound can be wrapped around the back of a user's leg and fastened at the front of his leg. Alternatively, the soft material wraparound can be wrapped around the front of a user's leg, and fastened at the back of his leg. Alternatively, a leg encircling arrangement can be constituted by a rigid elongated C-shaped thigh member for encircling the back of a user's thigh and a rigid elongated C-shaped calf member for encircling the back of a user's calf. The rigid elongated C-shaped thigh member and the rigid elongated C-shaped knee member are intended to be strapped onto a user's leg at the front of a user's leg. Yet alternatively, a leg encircling arrangement can include a semi-rigid semi-cylindrical open framework for encircling the front of a user's thigh and calf and intended to be manually pulled onto a user's leg before being strapped thereon, and reversibly removed from a user's leg. The outside leg triple anchor arrangement and/or inside leg triple anchor arrangement are suitably mounted on the different leg encircling arrangements or integrally manufactured therewith.

Knee braces of the present invention can be manufactured from a wide range of natural materials and/or synthetic materials suitable for direct contact with a user's leg. Knee braces of the present invention can be designed for fitting on a user's either leg. Alternatively, knee braces can be designed for fitting on a user's left leg or right leg. Knee braces can be typically provided in different sizes: Small, Medium, Large and Extra Large.

Knee braces of the present invention can be worn for assisting a user to straighten his leg, for example, in a squat position, climbing stairs, and the like, and enabling a user to perform leg flexion/extension exercises. The knee braces can also assist a user to sit down from a standing position in a controlled manner as opposed to suddenly collapsing.

### BRIEF DESCRIPTION OF DRAWINGS

In order to understand the present invention and to see how it can be carried out in practice, preferred embodiments will now be described, by way of non-limiting examples only, with reference to the accompanying drawings in which similar parts are likewise numbered.

FIG. 1A shows a pictorial view of a first embodiment of a knee brace in accordance with the present invention.

FIG. 1B is a pictorial view of a second embodiment of a knee brace in accordance with the present invention.

FIG. 1C is a pictorial view of a third embodiment of a knee brace in accordance with the present invention.

FIG. 2A is a pictorial view of a first bending resistance member pair, a bending resistance rod pair, a first stretch resistance member pair and a second stretch extension exercise member pair in accordance with the present invention.

FIG. 2B is a pictorial view of a second bending resistance member pair in accordance with the present invention.

FIG. 2C is a pictorial view of a second bending resistance member having a detachable proximal bending resistance section for enabling sliding insertion of a bending resistance rod.

FIG. 3 is a pictorial front elevation of an exterior wraparound surface of the FIG. 1A knee brace in an open state.

FIG. 4 is a pictorial front elevation of an interior wraparound surface of the FIG. 1A knee brace in an open state.

FIG. 5 is a pictorial perspective view of an outside leg thigh anchor of the FIG. 1A knee brace.

FIG. 6 is a pictorial perspective view of an outside leg calf anchor of the FIG. 1A knee brace.

FIG. 7 is a pictorial perspective view of an inside leg thigh anchor of the FIG. 1A knee brace.

FIG. 8 is a pictorial perspective view of an inside leg calf anchor of the FIG. 1A knee brace.

FIG. 9A is a pictorial right elevation of a straight outside leg strut of the FIG. 1A knee brace.

FIG. 9B is a pictorial front elevation of a straight outside leg strut of the FIG. 1A knee brace.

FIG. 10A is a pictorial right elevation of a flexed outside leg strut of the FIG. 1A knee brace.

FIG. 10B is a pictorial front elevation of a flexed outside leg strut of the FIG. 1A knee brace.

FIG. 11 is an exploded view of an outside leg strut of the FIG. 1A knee brace.

FIG. 12A is a pictorial left elevation of a straight inside leg strut of the FIG. 1A knee brace.

FIG. 12B is a pictorial front elevation of a straight inside leg strut of the FIG. 1A knee brace.

FIG. 13A is a pictorial left elevation of a flexed inside leg strut of the FIG. 1A knee brace.

FIG. 13B is a pictorial front elevation of a flexed inside leg strut of the FIG. 1A knee brace.

FIG. 14 is an exploded view of an inside leg strut of the FIG. 1A knee brace.

FIG. 15 is a pictorial front elevation of the exterior wraparound surface of the FIG. 1A knee brace including the FIG. 2A bending resistance member pair in a Ready-To-Use (RTU) state.

FIG. 16A is a pictorial front elevation of the exterior wraparound surface of the FIG. 1A knee brace including the FIG. 2B bending resistance member pair in a Ready-To-Use (RTU) state.

FIG. 16B is a pictorial view of the FIG. 16A knee brace strapped on a user's flexed leg.

FIG. 17A is a pictorial view of the FIG. 15 knee brace strapped on a user's straightened leg.

FIG. 17B is a pictorial view of the user's leg after flexing his leg.

FIG. 18A is a pictorial view of the FIG. 15 knee brace strapped on a user's flexed leg.

FIG. 18B is a pictorial view of the user's straightened leg.

FIG. 19A is a pictorial view of the FIG. 15 knee brace strapped on a user's flexed leg before application of an extension force for performing an extension exercise.

FIG. 19B is a pictorial view of the user's leg after application of the extension force.

FIG. 20 is a pictorial view showing a user wearing a knee brace pair and a drop foot prevention device.

FIG. 21 is a pictorial close-up view of the FIG. 20 drop foot prevention device.

FIG. 22 is a pictorial left elevation showing the use of the drop foot prevention device.

### DETAILED DESCRIPTION OF DRAWINGS

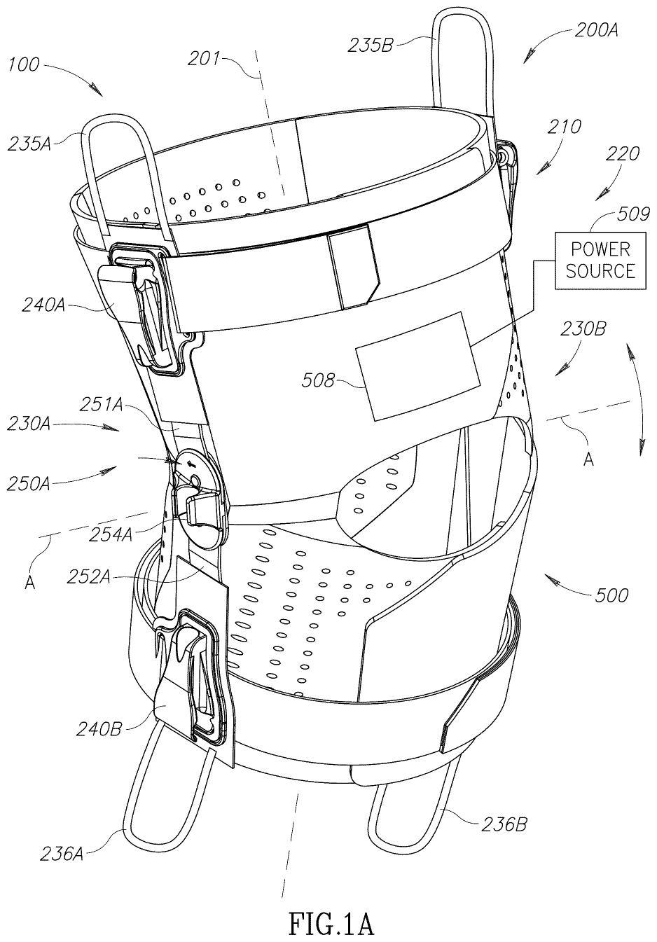

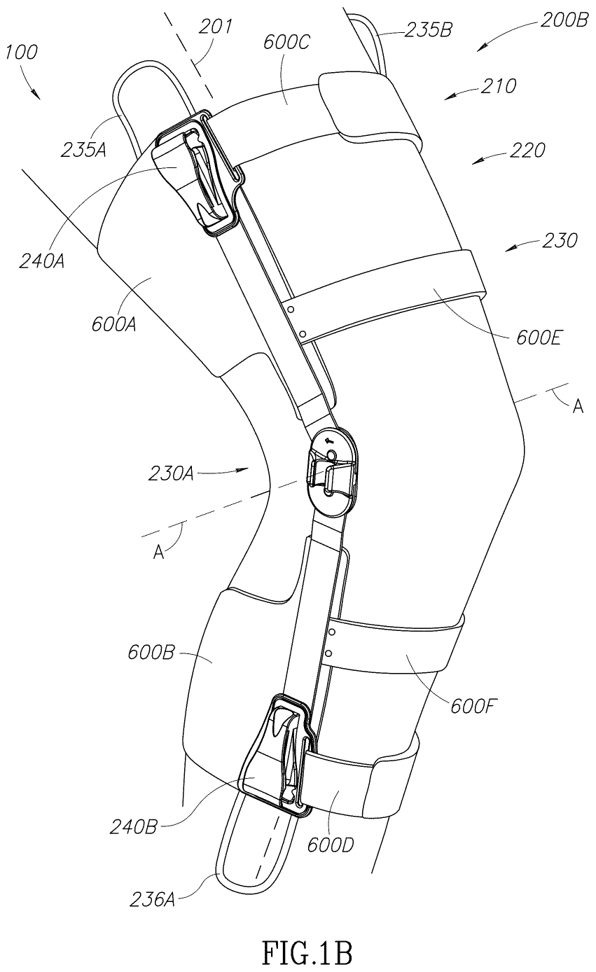

FIG. 1A to FIG. 1C, and FIG. 2A to FIG. 2C show a multi-purpose knee brace kit 100 includes a knee brace for strapping on a user's leg and accessories for use therewith. The knee brace can be alternatively implemented as a knee brace 200A (see FIG. 1A), a knee brace 200B (see FIG. 1B), a knee brace 200C (see FIG. 1C), and the like. The knee braces 200A-200C have a longitudinal knee brace centerline 201 and are intended to be flexed around a knee's flexion/extension axis A. The knee braces 200A-200C are designed for enabling a user to flex his leg between being straight and fully flexed. The accessories can be implemented as an initially generally straight bending resistance member pair 300A and 300B, a bending resistance rod pair 302A and 302B, and a stretch resistance member pair 400A and 400B (see FIG. 2A) and an initially generally straight bending resistance member pair 305A and 305B (see FIG. 2B).

The knee braces 200A-200C include a leg encircling arrangement 210 for at least partially encircling a user's at least thigh and calf, and a leg fastening arrangement 220 mounted on the leg encircling arrangement 210 for strapping the leg encircling arrangement 210 on a user's leg. The knee braces 200A-200C have different leg encircling arrangements 210 and different leg fastening arrangements 220 depending on their leg encircling arrangements 210. The leg fastening arrangements 220 are typically implemented by a multitude of Velcro straps, tie laces, clips, and the like.

The knee braces 200A-200C preferably include an opposite pair of pull loops 235A and 235B adjacent a user's thigh for assisting locating the knee braces 200A-200C on a user's leg. The opposite pair of pull loops 235A and 235B can also be employed for preventing downward slippage of a knee brace 200 by means of a user tying straps from a waistbelt to the opposite pair of pull loops 235A and 235B.

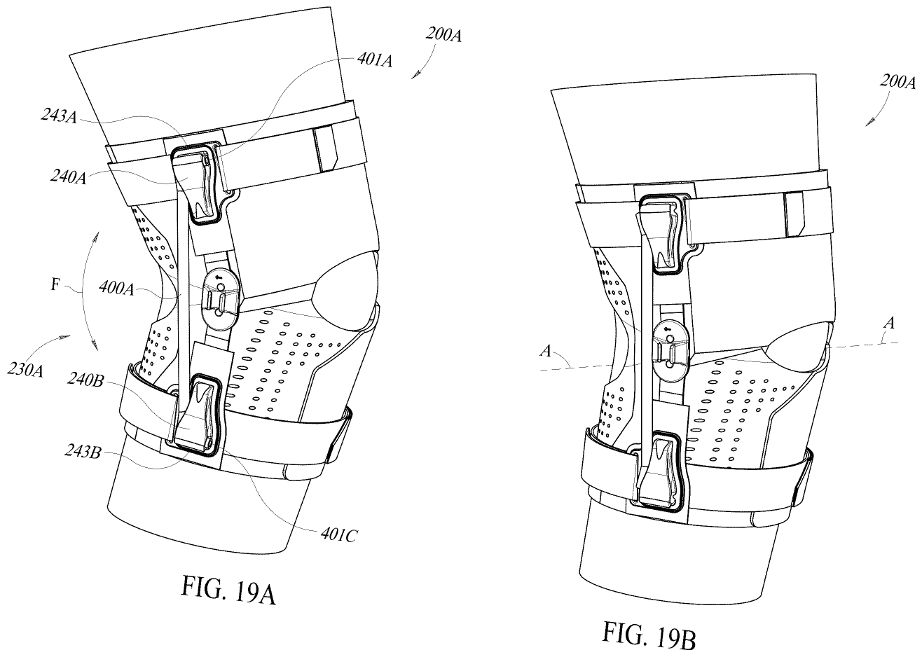

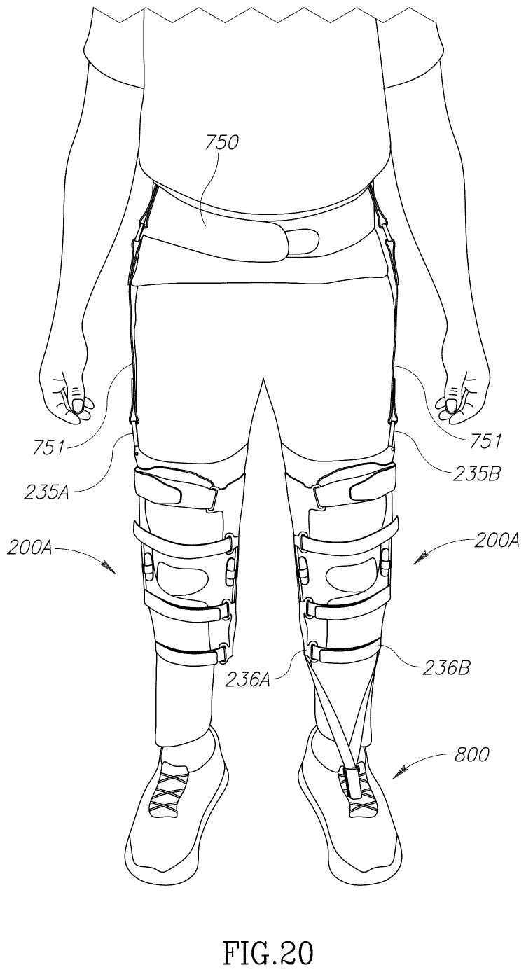

The knee braces 200A-200C preferably include an opposite pair of downwardly directed loops 236A and 236B for use with a drop foot prevention device 800 (see FIG. 20, FIG. 21A and FIG. 21B).

The knee braces 200A-200C are manufactured from a wide range of antibacterial natural and/or synthetic materials suitable for directly wearing on a user's leg. The terms “proximate” and “distal” refer to positions of components after strapping a knee brace 200 on a user's leg where proximate is towards a user's torso and distal is away from a user's torso. The knee braces 200A-200C are intended to be strapped on a user's left leg or right leg. For the illustrative purposes, the knee braces 200A-200C are described with reference to being strapped on a user's right leg. Accordingly, some components are described as being outside leg components and other components are described as being inside leg components.

The knee braces 200A-200C include an outside leg triple anchor arrangement 230A and an inside leg triple anchor arrangement 230B (not shown) mounted on the leg encircling arrangement 210. The outside leg triple anchor arrangement 230A includes an opposite pair of a thigh anchor 240A and a calf anchor 240B, and a strut 250A. The strut 250A includes a thigh strut member 251A, a calf strut member 252A, and a strut anchor 253A between the thigh strut member 251A and the calf strut member 252A for deployment at a knee's flexion/extension axis A. The strut anchor 253A includes a bending resistance member holder 254A. The inside leg triple anchor arrangement 230B has a similar construction as the outside leg triple anchor arrangement 230A.

FIG. 1A shows the knee brace 200A includes a leg encircling arrangement 210 constituted by a generally rectangular, generally planar knee brace wraparound 500 manufactured from a commercially available synthetic fabric material, for example, neoprene, and the like. The knee brace 200A is intended to be placed at the back of a user's leg and wrapped round the front of his leg before being strapped by the leg fastening arrangement 220.

FIG. 1B shows a knee brace 200B including a leg encircling arrangement 210 having a rigid elongated C-shaped thigh member 600A for encircling the back of a user's thigh and a rigid elongated C-shaped calf member 600B for encircling the back of a user's calf from his outside leg to his inside leg. The rigid elongated C-shaped thigh member 600A and the rigid elongated C-shaped calf member 600B are preferably suitably padded for a user's comfort. The leg fastening arrangement 220 includes a thigh fastening strap 600C and a calf fastening strap 600D for correspondingly fastening the rigid elongated C-shaped thigh member 600A and the rigid elongated C-shaped calf member 600B at the front or side of a user's leg. The knee brace 200B preferably includes an additional thigh fastening member 600E and an additional calf fastening member 600F for correspondingly fastening the rigid elongated C-shaped thigh member 600A and the rigid elongated C-shaped calf member 600B at the front or side of a user's leg.

The outside leg triple anchor arrangement 230A and the inside leg triple anchor arrangement 230B can be suitably mounted on the leg embracing arrangement 210. Suitable mounting techniques include riveting, gluing, ultrasonic welding, and the like. Alternatively, the leg embracing arrangement 210, the outside leg triple anchor arrangement 230A and the inside leg triple anchor arrangement 230B can be manufactured as a single monolithic construction.

FIG. 1C shows the knee brace 200C including a leg encircling arrangement 210 in the form of a semi-rigid semi-cylindrical open framework 700 for encircling the front of a user's thigh and calf. The knee brace 200C is intended to be manually pulled onto a user's leg and strapped thereon, and reversibly removed therefrom. The outside leg triple anchor arrangement 230A and the inside leg triple anchor arrangement 230B can be suitably mounted on the semi-rigid semi-cylindrical open framework 700. Suitable mounting techniques include riveting, gluing, ultrasonic welding, and the like. Alternatively, the semi-rigid semi-cylindrical open framework 700, the outside leg triple anchor arrangement 230A and the inside leg triple anchor arrangement 230B can be manufactured as a single monolithic construction.

FIG. 2A shows an initially generally straight bending resistance member pair 300A and 300B for replaceable mounting on the outside leg triple anchor arrangement 230A and the inside leg triple anchor arrangement 230B. The bending resistance member pair 300A and 300B have an unstretched straight length L1 and each terminate at opposite bending resistance member free end pair 301A and 301B. Each bending resistance member 300 typically has a bending stiffness in the range of 1 kg to 80 kg. The knee brace kit 100 can include two or more bending resistance member pairs having different bending stiffnesses. Each bending resistance member 300 can be constituted by an extension spring, a longitudinally directed leaf spring, a shape memory rod, and the like. The multi-purpose knee brace kit 100 optionally includes a bending resistance rod pair 302A and 302B for sliding insertion into the bending resistance member pair 300A and 300B for increasing their bending stiffness. The bending resistance rod pair 302A and 302B can be formed from suitable shape memory materials.

FIG. 2A shows a stretch resistance member pair 400A and 400B for replaceable mounting on the outside leg triple anchor arrangement 230A and the inside leg triple anchor arrangement 230B typically instead of the bending resistance member pair 300A and 300B. Each stretch resistance member 400 can be constituted by an extension spring, a stretch resistance band, a latex resistance tube, and the like, as used in training centers, rehabilitation centers, etc. In the case of the stretch resistance member pair 400A and 400B being constituted by stretch resistance bands, latex resistance tubes, and the like, the stretch resistance member pair 400A and 400B terminate at an opposite resistance member free end pair 401A and 401B typically constituted by wide diameter stoppers. The stretch resistance member pair 400A and 400B have an unstretched straight length L2. L2 can be the same length as L1 or preferably slightly shorter. Each stretch resistance member 400 typically has a stretch resistance in the range of 1 kg to 50 kg.

FIG. 2A shows the multi-purpose knee brace kit 100 optionally includes another stretch resistance member pair 402A and 402B having a greater stretch resistance than the stretch resistance member pair 400A and 400B thereby requiring a greater extension force to stretch the stretch resistance member pair 402A and 402B than the strength resistance member pair 400A and 400B to the same length.

FIG. 2B shows the multi-purpose knee brace kit 100 optionally includes an initially generally straight bending resistance member pair 305A and 305B also having an unstretched straight length L1. Each bending resistance member 305 includes a proximal bending resistance section 306, a central bending resistance section 307 and a distal bending resistance section 308. The central bending resistance section 307 can be constituted by an extension spring, a longitudinally directed leaf member, a shape memory rod, and the like. The proximal bending resistance section 306 and the distal bending resistance section 308 can be optionally constituted by solid rods which are not intended to bend.

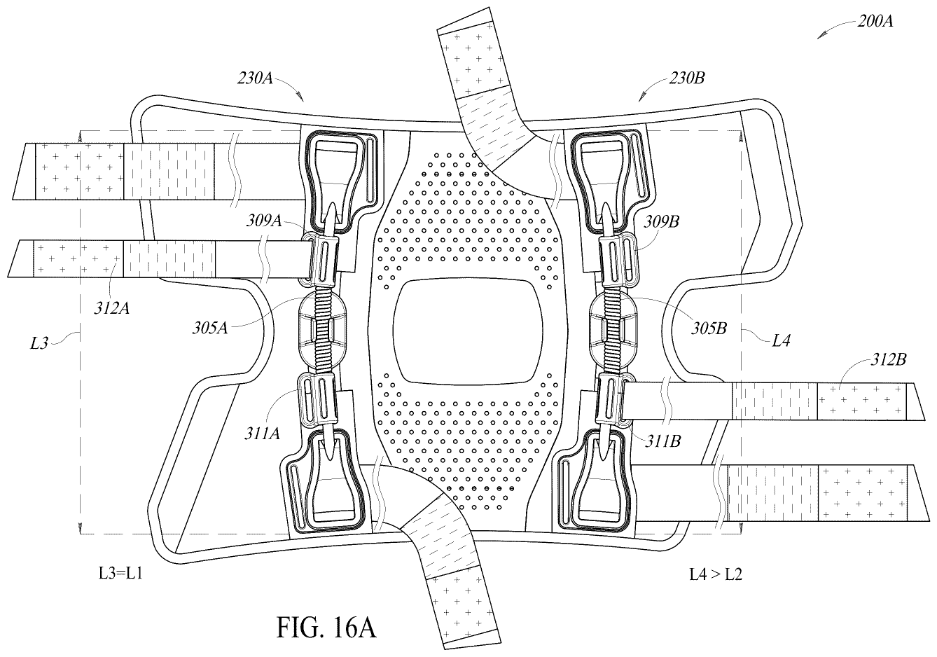

The proximal bending resistance section 306A preferably includes a fastening strap holder 309A and the distal bending resistance section 308A preferably includes a fastening strap holder 311A. The proximal bending resistance section 306B preferably includes a fastening strap holder 309B and the distal bending resistance section 308B preferably includes a fastening strap holder 311B. The fastening strap holder 311A preferably includes a fastening strap 312A for tethering the bending resistance member 305B to the bending resistance member 305A. Similar, the fastening strap holder 309B preferably includes a fastening strap 312B for tethering the bending resistance member 305A to the bending resistance member 305B. Tethering of the bending resistance member 305A and the bending resistance member 305B assists in maintaining correct placement of a knee brace 200 during use and particularly a knee brace 200A. FIG. 16A and FIG. 16B shows the knee brace 200A fitted with the bending resistance member pair 305A and 305B.

FIG. 2C shows a bending resistance member 305 preferably has a proximal bending resistance section 306 for screw insertion into a central bending resistance section 307 for enabling sliding insertion of a bending resistance rod 313 thereinto similar to a sliding insertion of the bending resistance rod 302 into a bending resistance member 300.

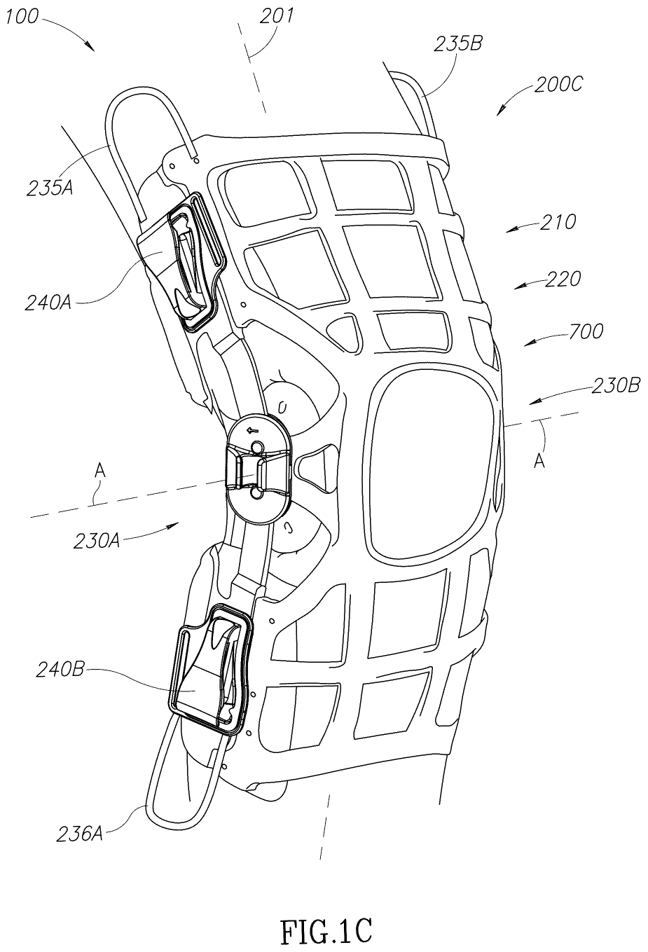

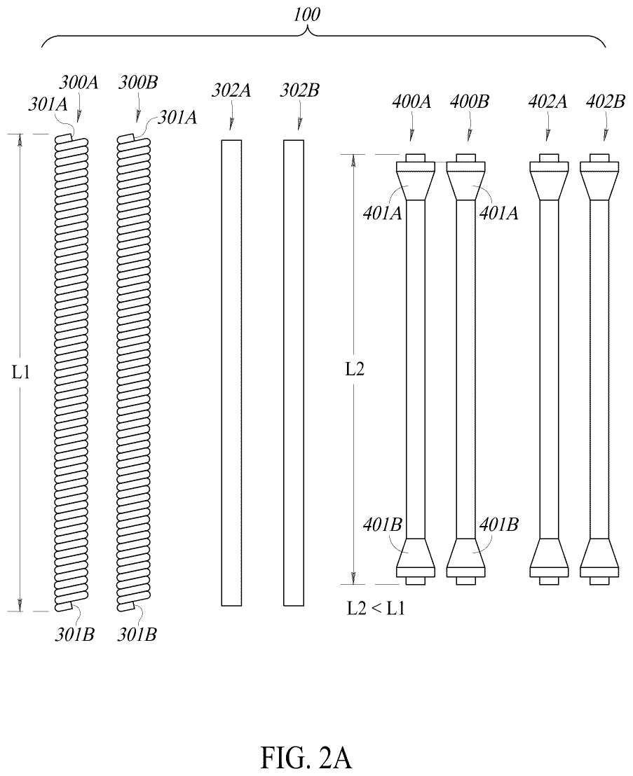

FIG. 3 and FIG. 4 show the wraparound 500 includes an exterior wraparound surface 501 facing away from a user's leg and an interior wraparound surface 502 facing towards a user's leg on strapping the knee brace 200A thereon. The interior wraparound surface 502 preferably has an anti-slip coating. The wraparound 500 includes an outside leg wraparound section 503, a central wraparound section 504 and an inside leg wraparound section 506. The wraparound 500 can be formed with a central throughgoing aperture 507 so that the back of a user's knee is exposed on wearing the knee brace 200A to facilitate its flexing and also enable air flow around a strapped knee. Alternatively, the central throughgoing aperture 507 can be provided with a breathable mesh. The knee brace 200A can be optionally fitted with a heating and/or cooling device 508 powered by a power source 509 (see FIG. 1A). Suitable heating/cooling devices 508 can include a Peltier device. The power source 509 can be electricity mains, a rechargeable battery, a button battery, and the like.

The exterior wraparound surface 501 includes an elongated outside leg thigh pocket 511 and an elongated outside leg calf pocket 512 opposite the outside leg thigh pocket 511 at the outside leg wraparound section 503. The elongated outside leg thigh pocket 511 is open towards a user's calf. The elongated outside leg calf pocket 512 is open towards a user's thigh. The exterior wraparound surface 501 includes an elongated inside leg thigh pocket 513 and an elongated inside leg calf pocket 514 opposite the inside leg thigh pocket 513 at the inside leg wraparound section 506. The elongated inside leg thigh pocket 513 is open towards a user's calf. The elongated inside leg calf pocket 514 is open towards a user's thigh.

The outside leg triple anchor arrangement 230A includes a thigh anchor 240A mounted on the outside leg thigh pocket 511, a calf anchor 240B mounted on the outside leg calf pocket 512 and an outside leg strut 250A. The inside leg triple anchor arrangement 230B includes a thigh anchor 240C mounted on the inside leg thigh pocket 513, a calf anchor 240D mounted on the inside leg calf pocket 514 and an inside leg strut 250B.

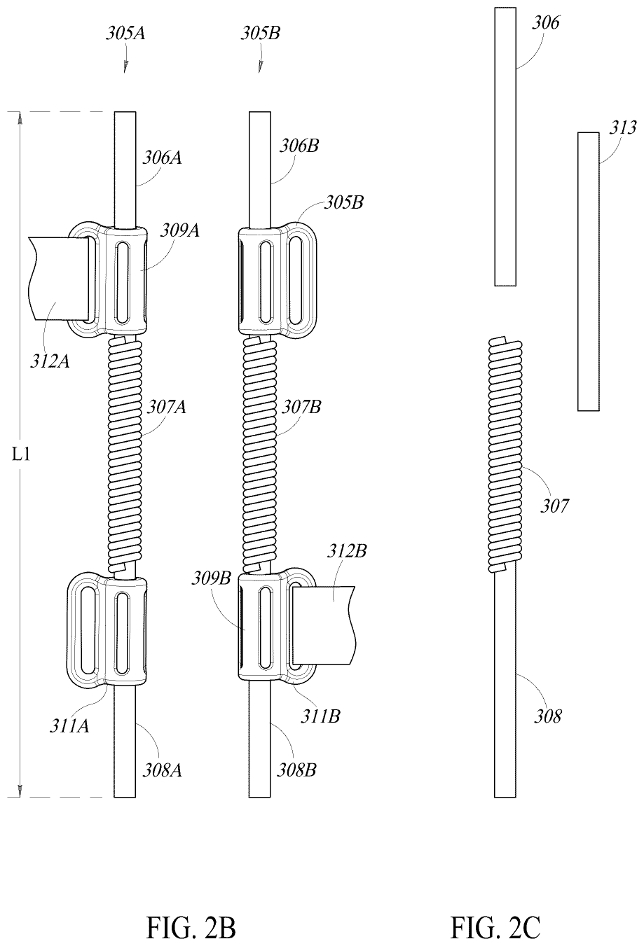

FIG. 5 shows the thigh anchor 240A includes a thigh anchor blind bore 241A open towards a user's calf, a thigh anchor transverse groove 242A proximal to the thigh anchor blind bore 241A and constituting a thigh fastening point for use with a stretch resistance member 400, and a thigh anchor elongated slot 243A constituting a part of the leg fastening arrangement 220.

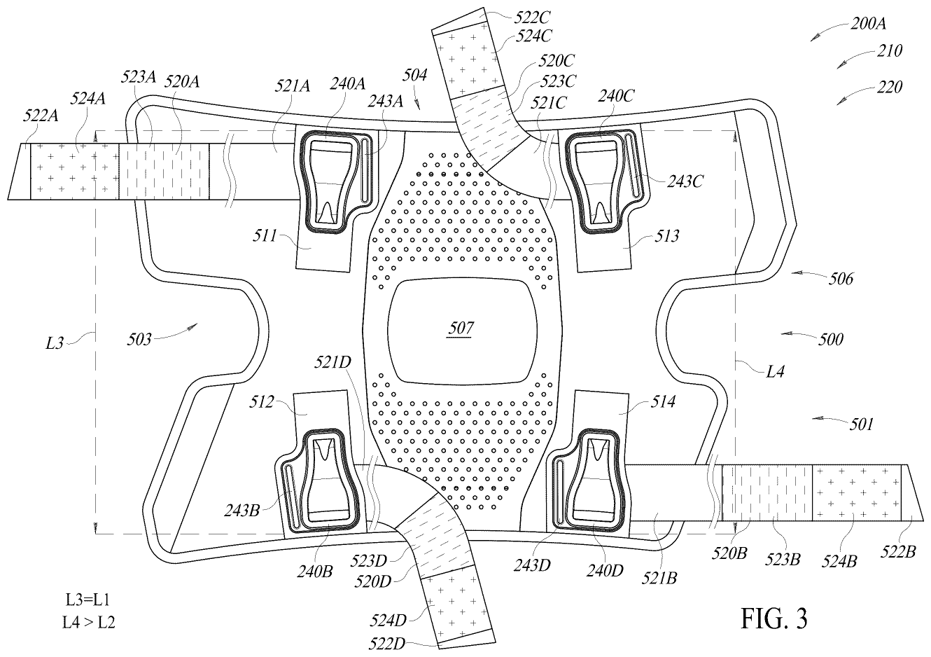

FIG. 6 shows the calf anchor 240B includes a calf anchor blind bore 241B open towards a user's thigh, a calf anchor transverse groove 242B distal to the calf anchor blind bore 241B and constituting a calf fastening point for use with a stretch resistance member 400, and a calf anchor elongated slot 243B constituting a part of the leg fastening arrangement 220.

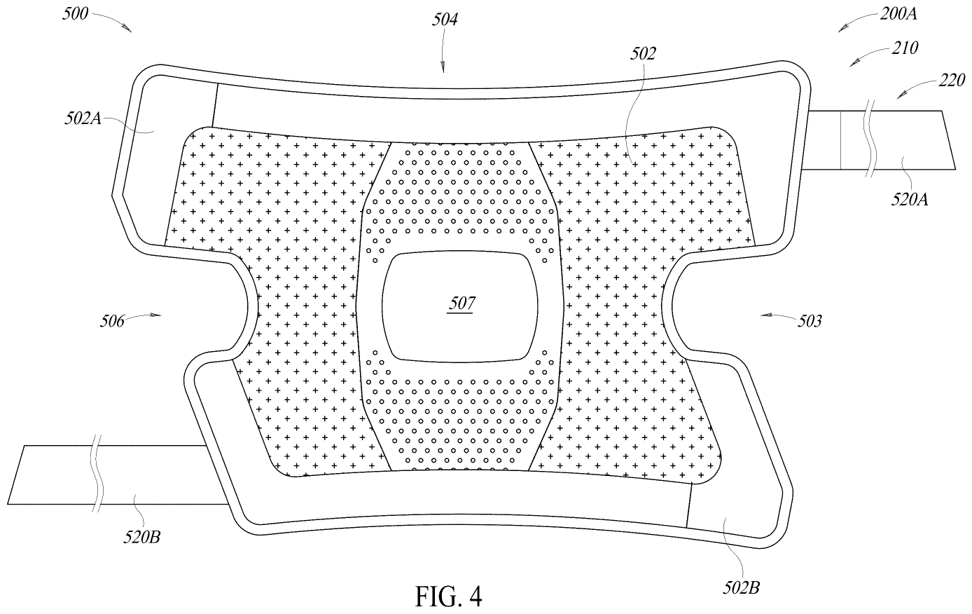

FIG. 7 shows the thigh anchor 240C includes a thigh anchor blind bore 241C open towards a user's calf, a thigh anchor transverse groove 242C proximal to the thigh anchor blind bore 241C and constituting a thigh fastening point for use with a stretch resistance member 400, and a thigh anchor elongated slot 243C constituting a component of the leg fastening arrangement 220.

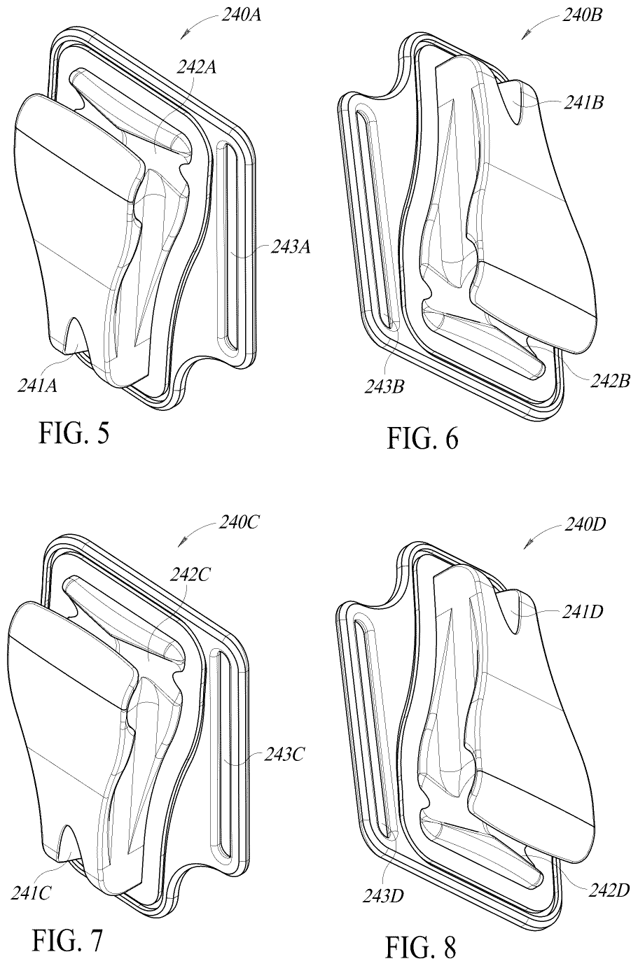

FIG. 8 shows the calf anchor 240D includes a calf anchor blind bore 241D open towards a user's thigh, a calf anchor transverse groove 242D distal to the calf anchor blind bore 241D and constituting a calf fastening point for use with a stretch resistance member 400, and a calf anchor elongated slot 243D constituting a component of the leg fastening arrangement 220.

The opposite pair of the thigh anchor blind bore 241A and the calf anchor blind bore 241B, and the opposite pair of the thigh anchor blind bore pair 241C and the calf anchor blind bore 241D each define a blind bore length L3 where L3=L1 for snugly receiving the initially generally straight bending resistance member pair 300A and 300B.

The opposite pair of thigh anchor transverse groove 242A and the calf anchor transverse groove 242B, and the opposite pair of the thigh anchor transverse groove 242C and the calf anchor transverse groove 242D each define a transverse groove pair length L4 where L4>L2 thereby requiring the stretch resistance member pair 400A and 400B to be stretched therebetween.

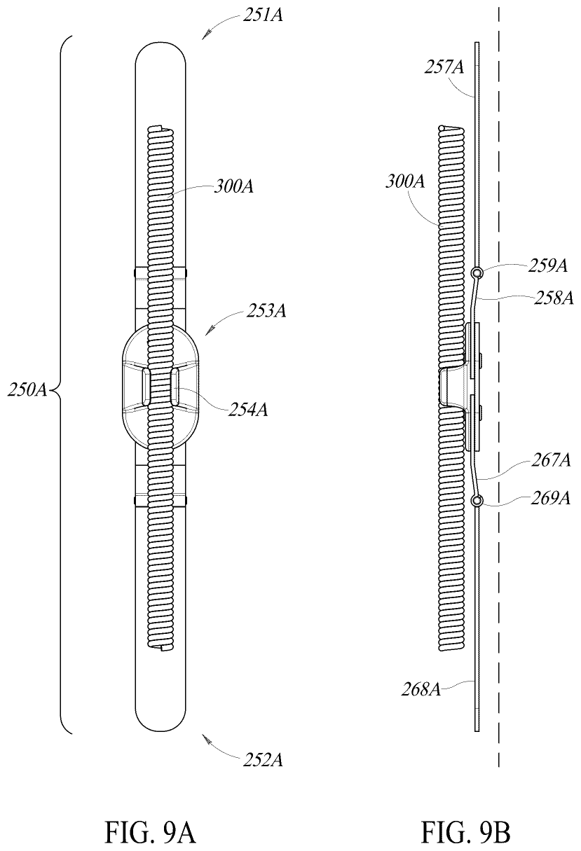

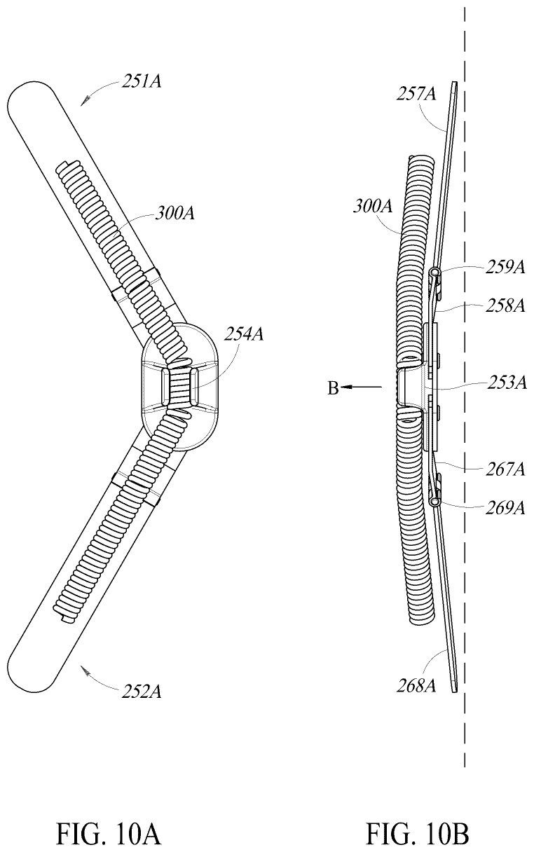

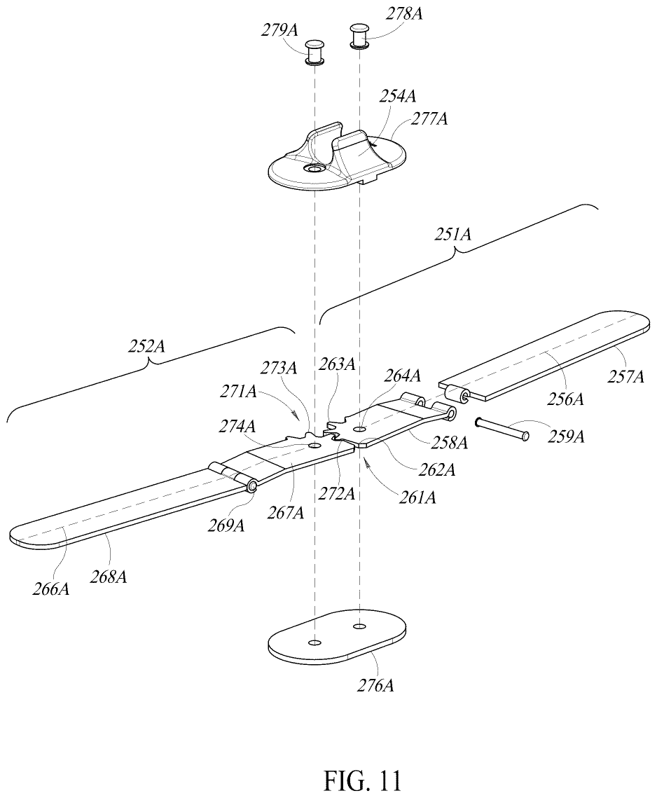

FIG. 9A and FIG. 9B, FIG. 10A and FIG. 10B, and FIG. 11 show the outside leg strut 250A includes the thigh strut member 251A, the calf strut member 252A and the strut anchor 253A with the bending resistance member holder 254A.

The thigh strut member 251A has a longitudinal thigh strut member centerline 256A and includes the following components: a proximal thigh strut member 257A, a distal thigh strut member 258A and a thigh strut hinge 259A transverse to the longitudinal thigh strut member centerline 256A such that the proximal thigh strut member 257A is pivotal to the distal thigh strut member 258A. The distal thigh strut member 258A terminates at a thigh strut member distal end 261A having a thigh strut member abutment surface 262A and adjacent thigh strut member cogs 263A. The distal thigh strut member 258A includes a thigh strut member throughgoing aperture 264A towards the thigh strut member distal end 261A.

The calf strut member 252A has a longitudinal calf strut member centerline 266A and includes the following components: a proximal calf strut member 267A, a distal calf strut member 268A and a calf strut hinge 269A transverse to the longitudinal calf strut member centerline 266A such that the distal calf strut member 268A is pivotal to the proximal calf strut member 267A. The proximal calf strut member 267A terminates at a calf strut member proximal end 271A having a calf strut member abutment surface 272A and adjacent calf strut member cogs 273A. The proximal calf strut member 267A includes a calf strut member throughgoing aperture 274A towards the calf strut member proximal end 271A.

The strut anchor 253A includes a strut anchor base 276A and a strut anchor cover 277A overlying the strut anchor base 276A. The strut anchor 253A includes a proximal rivet 278A for mounting the strut anchor cover 277A on the strut anchor base 276A via the thigh strut member throughgoing aperture 264A. The strut anchor 253A includes a distal rivet 279A for mounting the strut anchor cover 277A on the strut anchor base 276A via the calf strut member throughgoing aperture 274A.

The outside leg strut 250A is mounted on the knee brace 200A by sliding insertion of the thigh strut member 251A into the outside leg thigh pocket 511 and the calf strut member 252A into the outside leg calf pocket 512.

In the case of a straight leg, the bending resistance member 300A is snugly accommodated within the bending resistance member holder 254A and the thigh strut member abutment surface 262A abuts against the calf strut member abutment surface 272A to avoid hyperextension (see FIG. 9A and FIG. 9B). The outside leg strut 250A including its strut anchor 253A are adjacent a user's outside leg denoted as the dashed line.

In the case of a user flexing his leg, the thigh strut member cogs 263A mesh with the calf strut member cogs 273A, and the bending resistance member 300A tends to move laterally away from a user's outside leg denoted as dashed line in order to reduce its extension. Accordingly, the bending resistance member 300A entrains the outside leg strut 250A therewith which tends to pivot the thigh strut member 251 at its thigh strut hinge 259A and the calf strut member 252A at its calf strut hinge 269A to slightly arch the strut anchor 253A laterally outwards from a user's right leg as denoted by arrow B to prevent the bending resistance member 300A from detaching itself from the bending resistance member holder 254A (see FIG. 10A and FIG. 10B).

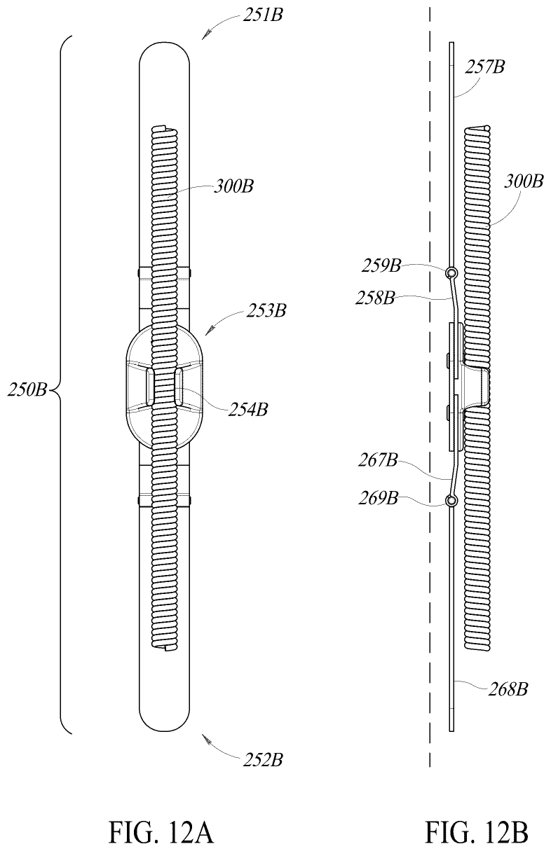

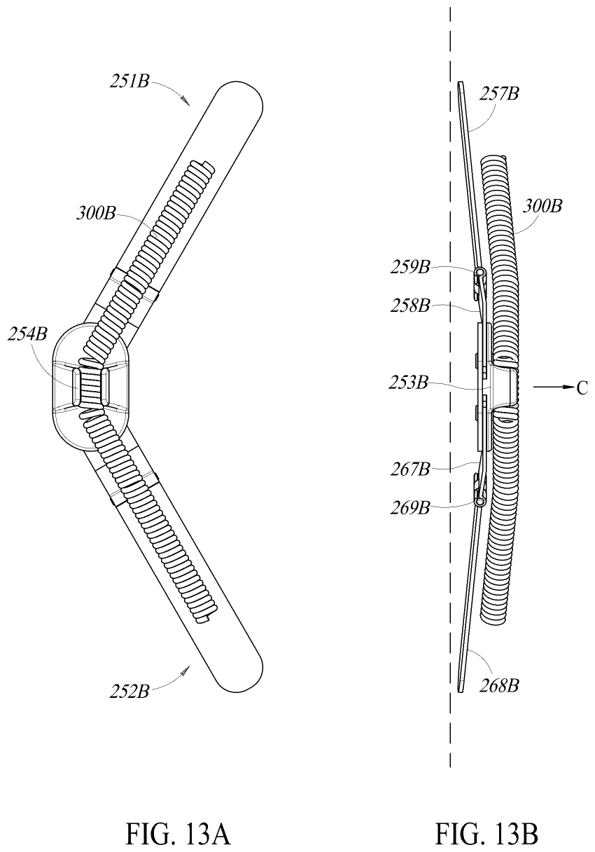

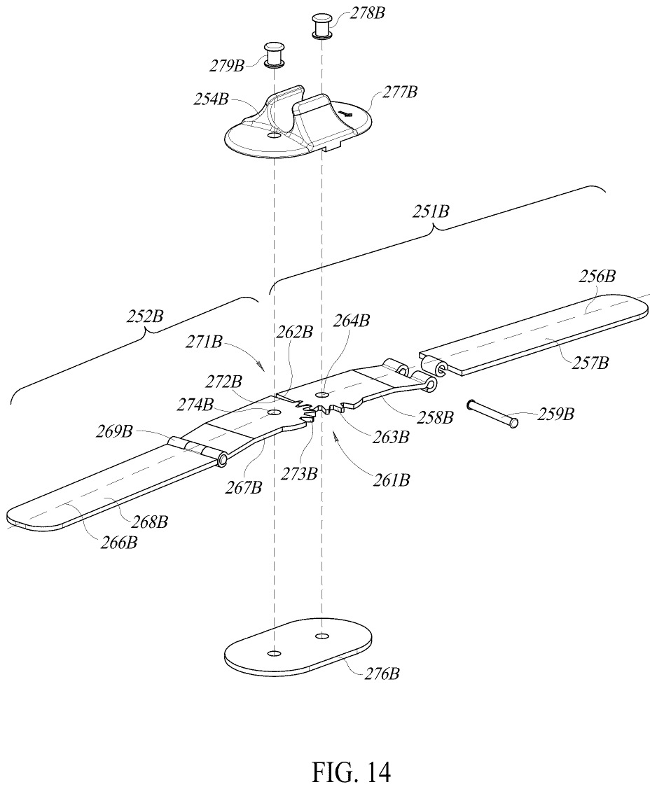

FIG. 12A and FIG. 12B, FIG. 13A and FIG. 13B, and FIG. 14 show the inside leg strut 250B has a similar construction as the outside leg strut 250A and therefore similar parts are likewise numbered. The inside leg strut 250B operates in a similar manner as the outside leg strut 250A on flexing the knee brace 200A except that the inside leg strut 250B arches slightly laterally inwards away from a user's right leg as denoted by arrow C.

The leg fastening arrangement 220 includes the following components:

The interior wraparound surface 502 has an upper inside leg corner 502A provisioned with Velcro-like hooks for fastening on the exterior wraparound surface 501 on wrapping the knee brace 200A around a user's leg.

The interior wraparound surface 502 has a lower outside leg corner 502B provisioned with Velcro-like hooks for fastening on the exterior wraparound surface 501 on wrapping the knee brace 200A around a user's leg.

The exterior wraparound surface 501 is provisioned with four widthwise extending fastening straps 520A-520D. The fastening straps 520A and 520B are intended to be strapped at the front of a user's leg. The fastening straps 520C and 520D are intended to be strapped at the back of a user's leg.

The upper fastening strap 520A has a first fastening strap end 521A securely attached to the exterior wraparound surface 501 at the outside leg thigh pocket 511 and a second fastening strap end 522A extending sidewards to be wrapped around a user's thigh. The fastening strap 520A includes a Velcro-like loop section 523A and terminates at a Velcro-like hook section 524A. The Velcro-like hook section 524A is threaded through the thigh anchor elongated slot 243C to be fastened onto the Velcro-like loop section 523A.

The lower fastening strap 520B has a first fastening strap end 521B securely attached to the exterior wraparound surface 501 at the inside leg calf pocket 514 and a second fastening strap end 522B extending sidewards to be wrapped around a user's thigh. The fastening strap 520B includes a Velcro-like loop section 523B and terminates at a Velcro-like hook section 524B. The Velcro-like hook section 524B is threaded through the calf anchor elongated slot 243B to be fastened onto the Velcro-like loop section 523B.

The upper fastening strap 520C has a first fastening strap end 521C securely attached to the exterior wraparound surface 501 at the inside leg thigh pocket 513 and a second fastening strap end 522C extending sidewards. The fastening strap 520C includes a Velcro-like loop section 523C and terminates at a Velcro-like hook section 524C. The Velcro-like hook section 524C is threaded through the thigh anchor elongated slot 243A to be fastened onto the Velcro-like loop section 523C.

The lower fastening strap 520D has a first fastening strap end 521D securely attached to the exterior wraparound surface 501 at the outside leg calf pocket 512 and a second fastening strap end 522B extending sidewards. The leg fastening strap 520D includes a Velcro-like loop section 523D and terminates at a Velcro-like hook section 524D. The Velcro-like hook section 524D is threaded through the calf anchor elongated slot 243D to be fastened onto the Velcro-like loop section 523D.

### Use of the Knee Brace Kit for Flexion/Extension and Other Uses

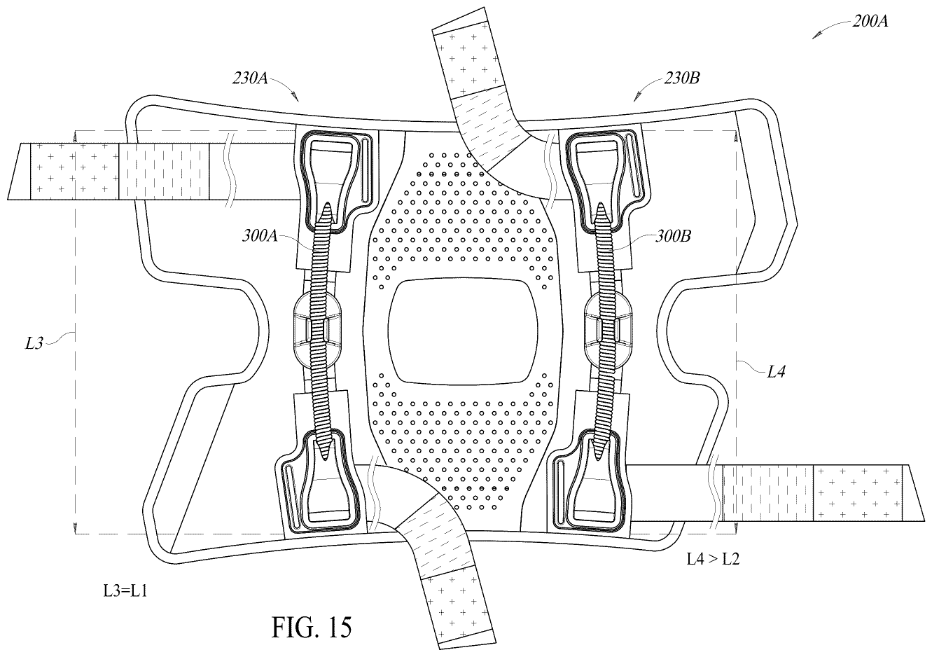

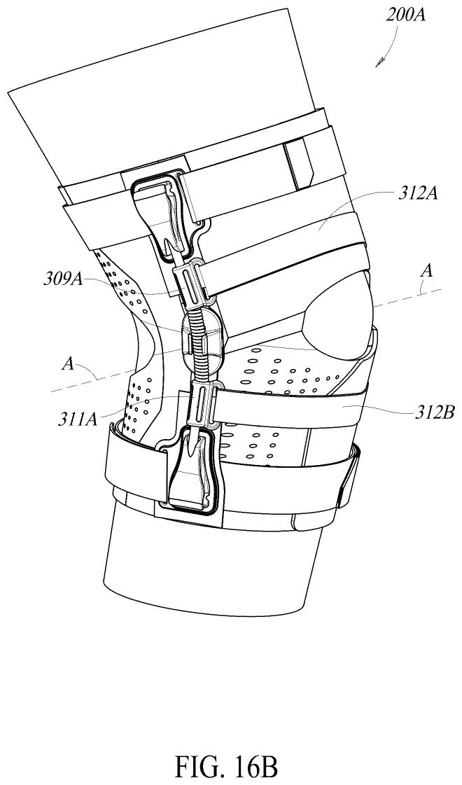

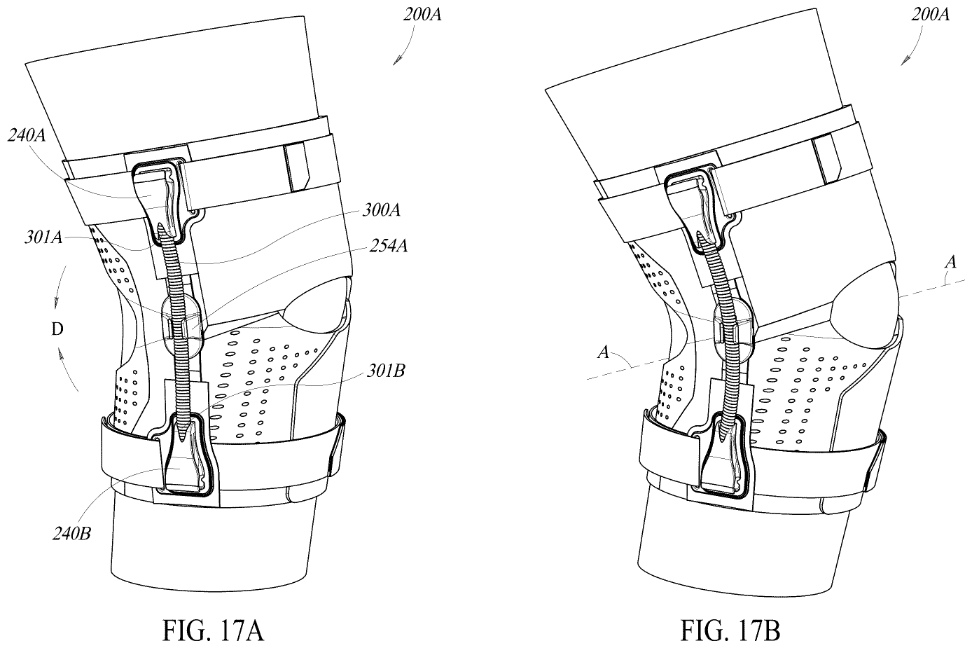

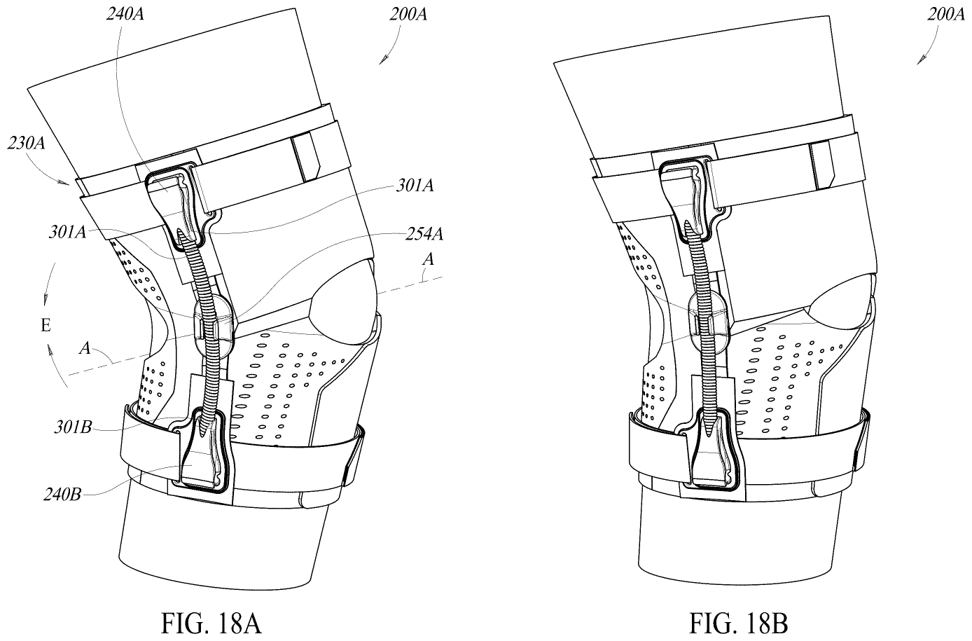

FIG. 15, FIG. 16A and FIG. 16B, FIG. 17A and FIG. 17B, FIG. 18A and FIG. 18B, and FIG. 19A and FIG. 19B show the use of the knee brace 200A as follows:

FIG. 15 shows assembly of the knee brace 200A to form a Ready-To-Use (RTU) knee brace 200A as follows: The user mounts the bending resistance member 300A on the outside leg triple anchor arrangement 230A by inserting its free end 301A into the thigh anchor 240A, its free end 301B into the calf anchor 240B, and its middle section into the central bending resistance member holder 254A. And similarly, the user mounts the bending resistance member 300B on the inside leg triple anchor arrangement 230B by inserting its free end 301A into the thigh anchor 240C, its free end 301B into the calf anchor 240D, and its middle section into the central bending resistance member holder 254B (not shown). A Ready-To-Use (RTU) knee brace 200A has a nominal straight shape by virtue of the initially generally straight bending resistance member pair 300A and 300B's length L1 being the same length as the blind bore pair length L3. The RTU knee brace 200A is placed at the back of a user's leg and is wrapped before being fastened at the front of the user's leg by the leg fastening arrangement 220. On wearing the knee brace 200A, the bending resistance member 300A is lateral to the strut 250A and the bending resistance member 300B is lateral to the strut 250B relative to the user's leg. Alternatively, the knee brace 200A can be fitted with the bending resistance member pair 305A and 305B instead of the bending resistance member pair 300A and 300B (see FIG. 16A and FIG. 16B).

The RTU knee brace 200A can be used for flexion exercises as follows: After a user straps the RTU knee brace 200A on his leg, the knee brace 200A urges the user to straighten his leg (see FIG. 17A). The user is required to apply a flexion force denoted by arrow D to flex the RTU knee brace 200A by bending the bending resistance member pair 300A and 300B at the knee flexion/extension axis A (see FIG. 17B). The user can deploy a single bending resistance member 300, thereby requiring less flexion force to flex the knee brace 200A. Alternatively, the user can insert the bending resistance rod pair 302A and 302B into the bending resistance member pair 300A and 300B for increasing the flexion force to flex the knee brace 200A.

If a user is squatting, climbing stairs, and the like, the user's weight bends the bending resistance member pair 300A and 300B to flex the knee brace 200A denoted by arrow E (see FIG. 18A). The bending resistance member pair 300A and 300B assist the user to straighten his leg by reverting to their initially generally straight shape, thereby affording the user having to exert less force (see FIG. 18B). The user can deploy a single bending resistance member 300 for less assistance. Alternatively, the user can insert the bending resistance rod pair 302A and 302B into the bending resistance member pair 300A and 300B for increasing the bending stiffness for increasing assistance.

FIG. 19A and FIG. 19B show a user's leg having a RTU knee brace 200A intended for extension exercises. The RTU knee brace 200A is assembled as follows: The user mounts the stretch resistance member 400A on the outside leg triple anchor arrangement 230A by stopping its stopper 401A in the thigh anchor transverse groove 242A and its stopper 401B in the calf anchor transverse groove 242B. And similarly, the user mounts the stretch resistance member 400B on the inside leg triple anchor arrangement 230B by stopping its stopper 401A in the thigh anchor transverse groove 242C and its stopper 401B in the calf anchor transverse groove 242D. The RTU knee brace 200A has a nominal flexed shape (see FIG. 19A) by virtue of the stretch resistance member pair 400A and 400B's unstretched length L2 being shorter than the transverse groove pair length L4.

After a user straps the RTU knee brace 200A on his leg, the RTU knee brace 200A urges the user to flex his leg at the knee flexion/extension axis A. The user is required to apply an extension force denoted by arrow F to straighten his leg (see FIG. 19B) by stretching the stretch resistance member pair 400A and 400B. The user can deploy a single stretch resistance member 400 for less resistance. Alternatively, the user can replace the stretch resistance member pair 400A and 400B by the stretch resistance member pair 402A and 402B for increasing the extension force to straighten the RTU knee brace 200A.

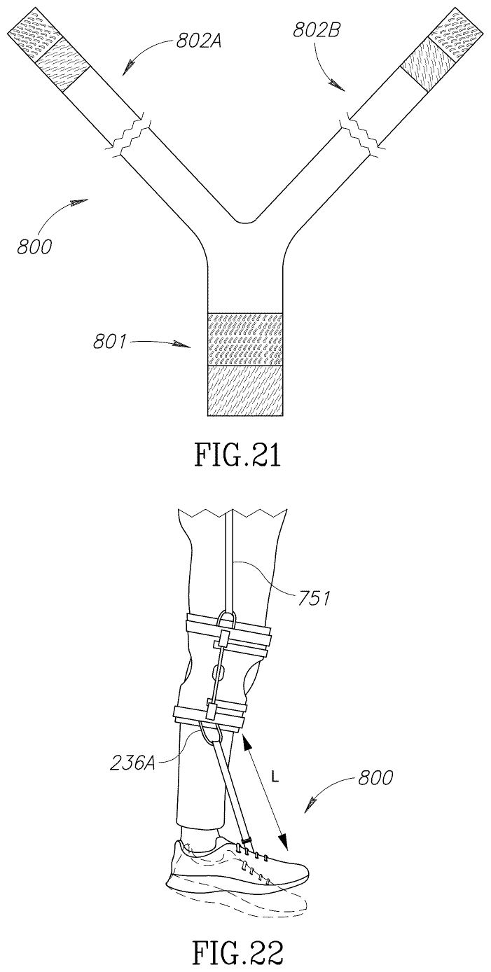

FIG. 20 shows a user wearing a knee brace pair 200A. The user preferably prevents downward slippage of the knee brace pair 200A by wearing an anti-slip waist belt 750 including two non-elastic straps 751 for fastening on the lateral pull loops 235A and 235B. FIG. 21 shows a drop foot prevention device 800 including a shoe fastening arrangement 801 for fastening to a shoe and a pair of non-elastic shoe tethers 802A and 802B. The fastening arrangement 801 and the shoe tethers 802, 802B can employ a range of fastening means including inter alia Velcro strips, snap fits, and the like. The shoe tethers 802A and 802B preferably have an adjustable length L such that the drop foot prevention device 800 is suitable for users of different heights. FIG. 22 shows the user deploying the drop foot prevention device 800 supported by the shoe tethers 802A and 802B being fastened to opposite downward depending loops 236A and 236B.

While the invention has been described with respect to a limited number of embodiments, it will be appreciated that many variations, modifications, and other applications of the invention can be made within the scope of the appended claims.

1. A knee brace kit for use by a user, the user's leg having a knee flexion/extension axis, the knee brace kit comprising:
a) a knee brace having a longitudinal knee brace centerline and including:
i) a leg encircling arrangement for encircling the user's at least thigh and calf,
ii) a leg fastening arrangement mounted on said leg encircling arrangement for strapping said leg encircling arrangement on the user's thigh and calf, and
iii) an at least one triple anchor arrangement of an outside leg triple point anchor arrangement and an inside leg triple anchor arrangement mounted on said leg encircling arrangement,
each said triple anchor arrangement includes an opposite pair of a thigh anchor and a calf anchor, and a strut,
a said thigh anchor including a thigh anchor blind bore facing the user's calf,
a said calf anchor including a calf anchor blind bore facing the user's thigh,
a said strut including:
a thigh strut member with a thigh strut member distal end having a thigh strut member abutment surface and adjacent thigh strut member cogs,
a calf strut member with a calf strut member proximal end having a calf strut member abutment surface and adjacent calf strut member cogs, and
a strut anchor between said thigh strut member distal end and said calf strut member proximal end for deployment at the knee's flexion/extension axis, said strut anchor including a bending resistance member holder; and
b) at least one initially generally straight bending resistance member configured for being removably mounted on a said triple anchor arrangement,
each said bending resistance member having a pair of opposite bending resistance member free ends configured for longitudinal sliding insertion into said opposite pair of said thigh anchor blind bore and said calf anchor blind bore, and insertion in a said bending resistance member holder,
whereby, in use on wearing a knee brace, each said bending resistance member is lateral to a said strut relative to a user's leg,
such that, in use, on a user straightening his knee, said thigh strut member abutment surface abuts said calf thigh strut member abutment surface, and on the user bending his knee, said thigh strut member cogs mesh with said calf strut member cogs.

2. The kit according to claim 1 wherein a said thigh strut member has a longitudinal thigh strut member centerline and includes a transverse hinge towards said thigh strut member distal end for enabling a pivot movement perpendicular to its said longitudinal thigh strut member centerline.

3. The kit according to claim 1 wherein a said calf strut member has a longitudinal calf strut member centerline and includes a transverse hinge towards the calf strut member proximal end for enabling a pivot movement perpendicular to its said longitudinal calf strut member centerline.

4. The kit according to claim 1 wherein said knee brace includes an opposite pair of pull loops adjacent a user's thigh for assisting location of said knee brace.

5. The kit according to claim 1 wherein said leg encircling arrangement includes a soft material wraparound configured for wrapping around the back of a user's at least thigh and calf and fastening at the front of the user's leg.

6. The kit according to claim 1 wherein said leg encircling arrangement includes a rigid elongated C-shaped thigh member configured for encircling the back of a user's thigh and fastening at the front of the user's leg and a rigid elongated C-shaped calf member configured for encircling the back of a user's calf and fastening at the front of the user's leg.

7. The kit according to claim 1 wherein said leg encircling arrangement includes a semi-rigid semi-cylindrical open framework for encircling the front of a user's thigh and calf and fastening at the back of the user's leg.

8. The kit according to claim 1 wherein an initially generally straight bending resistance member pair includes fastening straps for tethering said an initially generally straight bending resistance member pair together at the front of a user's leg.

9. The kit according to claim 1 wherein a bending resistance member is configured to slidingly accommodate a replaceable bending resistance rod therein for increasing its bending stiffness.

10. The kit according to claim 1 and further comprising at least one stretch resistance member terminating at an opposite stretch resistance member free end pair being constituted by a wide diameter stopper pair, and
wherein said knee brace further includes
a thigh fastening point proximal to said thigh anchor for removably fastening one stretch resistance member free end, and
a calf fastening point distal to said calf anchor for removably fastening the other stretch resistance member free end whereby, on a user straightening his leg, said stretch resistance member being stretched mounted between said thigh fastening point and said calf fastening point.

11. The kit according to claim 10 wherein
a said thigh anchor includes a thigh anchor transverse groove proximal to said thigh anchor blind bore wherein said thigh anchor transverse groove constitutes said thigh fastening point and
a said calf anchor includes a calf anchor transverse groove distal to said calf anchor blind bore wherein said calf anchor transverse groove constitutes said anchor fastening point.

12. The kit according to claim 1 and further comprising a drop foot prevention device including a shoe fastening arrangement and a pair of shoe tethers for fastening to opposite sides of said knee brace.
